(19) United States
(12) Patent Application Publication
(10) Pub. No.: US 2026/0351397 A1
(43) Pub. Date: Nov. 5, 2026

(54) CLINICAL IDLE-MODE DETECTOR FOR MEDICAL DEVICE MAINTENANCE FUNCTIONS

(71) Applicant: KONINKLIJKE PHILIPS N.V., EINDHOVEN (NL)

(72) Inventor: Robert Paul KOSTER, EINDHOVEN (NL)

(21) Appl. No.: 19/125,336

(22) PCT Filed: Oct. 23, 2023
(86) PCT No.: PCT/EP2023/079396

§ 371 Date: Apr. 29, 2025

Related U.S. Application Data
(60) Provisional application No. 63/422,086, filed on Nov. 3, 2022.

(51) Int. Cl. G16H 40/40

(52) U.S. Cl. CPC G16H 40/40

(57) ABSTRACT

A non-transitory computer readable medium stores instructions readable and executable by at least one electronic processor (14) to perform a method of governing automated maintenance of a medical device (10). The method includes determining at least one status indicator of a medical device; determining, from the at least one status indicator, an idle state of the medical device; and when the idle state of the medical device is idle, performing a maintenance function on the medical device.

### FIELD

The following relates generally to the medical device maintenance arts, medical device security validation arts, and related arts.

### BACKGROUND

Maintenance monitoring is rapidly evolving from typical information technology (IT) to also include medical devices and medical networks to support the secure and safe operation thereof. Maintenance monitoring can refer to, for example, a security monitor that applies passive network monitoring to monitor network traffic that can detect security vulnerabilities and ongoing attacks on the network or active network scanning to identify equipment on the network. In another example, maintenance monitoring can be input to risk assessment and vulnerability identification (e.g., because identified equipment is vulnerable). Maintenance monitoring can refer to, for example, a maintenance monitor that monitors potential issues with medical devices that can result in poor performance or potential downtime. Maintenance functions can include network security monitoring, network security scanning, log collection and upload, incremental backups, software integrity scans, medical device configuration checks, and/or so forth.

In the IT security space, micro segmentation in hospital networks is on the rise to strengthen security. However, this makes it more costly and/or complex to perform security monitoring. A logical extension is to leverage computerized medical devices to perform IT security monitoring. This provides the benefit that the medical devices are already present on the IT network and at the right location. An additional benefit is the ability to monitor security properties that are of particular interest for medical devices.

However, for patient safety, medical devices are subject to strict benefit-risk assessments and validation requirements to ensure the clinical function is not negatively affected. This poses a challenge for security monitoring (and/or other types of maintenance monitoring) functionality on medical devices because such function may compete for computing resources (CPU, memory, network bandwidth, and so forth), which is further complicated because security monitoring functionality may be subject to shorter software lifecycles than the clinical software. This could for example include updates of monitoring profiles, filtering patterns and alert signatures. These updates may affect the system in unpredictable ways, e.g., increased system resource usage.

The following discloses certain improvements to overcome these problems and others.

### SUMMARY

In one aspect, a non-transitory computer readable medium stores instructions readable and executable by at least one electronic processor to perform a method of governing automated maintenance of a medical device. The method includes determining at least one status indicator of a medical device; determining, from the at least one status indicator, an idle state of the medical device; and when the idle state of the medical device is idle, performing a maintenance function on the medical device.

In another aspect, a medical device includes a network interface operative to connect the medical device with an electronic data network, an electronic processor, and a non-transitory storage medium storing instructions readable and executable by the electronic processor to implement processing modules including a medical function module operative to control the medical device to perform patient medical care; at least one maintenance module operative to perform at least one maintenance function on the medical device utilizing the network interface; an idle assessment module operative to determine whether the medical device is idle or is performing patient medical care; and an application programming interface (API) interfacing the idle assessment module and the at least one maintenance module to control the at least one maintenance module to perform the at least one maintenance function when the medical device is idle and not when the medical device is performing patient medical care.

In another aspect, a method of governing automated maintenance of a medical device includes determining at least one status indicator of a medical device; determining, from the at least one status indicator, an idle state of the medical device, wherein the idle state is idle when the medical device is not performing patient medical care and is not idle when the medical device is performing patient medical care; and preventing a maintenance function on the medical device from being performed when the idle state of the medical device is not idle.

One advantage resides in ensuring that background maintenance functions of a medical device do not interfere with patient care procedures using the same medical device.

Another advantage resides in determining an idle state or a not-idle state of the medical device.

Another advantage resides in estimating a time interval for when a medical device will be in an idle state to ensure that maintenance procedures can be performed within the estimated time interval.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for governing automated maintenance of a medical device in accordance with the present disclosure.

FIG. 2 diagrammatically illustrates an illustrative method for governing automated maintenance of a medical device in accordance with the present disclosure.

### DETAILED DESCRIPTION

Computerized medical devices such as medical imaging devices and patient monitors are connected with an information technology (IT) network, and can benefit from running various maintenance functions such as machine log upload functions and/or security functions, for example network security monitoring or scanning to detect anomalous network traffic potentially indicative of malicious threats such as bot networks or device communication misconfigurations, software integrity scan, performing incremental backups, and so forth. In some settings, the IT network may be a task-specific dedicated network such as a radiology network or a patient monitoring network, and in such cases the medical devices on the network could also be beneficially leveraged to perform IT network-wide security functions as well. Medical devices can also have security functions that are domain-specific, for example a security function that detects a misconfiguration of the medical device that could adversely impact patient care.

However, due to the life-critical nature of some types of medical care provided by these medical devices and to assure patient safety, maintenance functions should be run only at times when the medical device is not being used to perform patient medical care. This can limit the time windows available for performing maintenance functions, which in turn reduces the frequency of running the maintenance functions with concomitant increase in vulnerabilities. For example, if an imaging device is misconfigured in the morning this might not be detected until the next night, or even the upcoming weekend, when the device is idle, and a suitable maintenance function can be run that detects the misconfiguration.

The following discloses facilitating implementation of maintenance functions executed by a medical device by providing an idle detector. This includes several components. A device status monitor continually detects the state or mode of the medical device, as measured by indicators such as the menu screen currently being displayed, the automatic log entries being generated, sensor readings of the medical device, and so forth.

Idle detection logic is applied to these indicators to assess whether the medical device is idle, or in some embodiments the level of idleness. In this context, “idle” can be variously defined but one useful definition is that the medical device is not performing any tasks that involve patient manipulation or patient data collection (e.g., operation of the patient support of a medical imaging device or acquisition of images or patient data in the case of a patient monitor would be “not idle” states). The “idle” state could also be secondarily defined as the medical device processing level being below some maximum threshold, i.e., even if the medical device is not interacting with a patient, it might be considered “non-idle” if it is running a computationally intensive process such as complex image processing.

When the medical device is assessed to be idle (i.e., not performing patient medical care), the idle detection logic may also estimate a likely timeframe of idleness. As one example, if the idle detection logic determines that no one is logged into the medical device (e.g., because the login screen is currently displayed) then the logic may estimate that the imaging device will remain idle for some fixed time interval that amounts to how long a user takes to log in and set up for the first patient. As another example, if the idle detection logic is implemented as a machine learning (ML) component then the artificial neural network (ANN) or other ML component can be trained on data to assess both the idle/non-idle state and the timeframe of idleness based on historical training data that provides examples of the time from a given idle state until the medical device exits the idle state.

An idle detector application program interface (API) enables maintenance functions to interface with the idle detector. The idle detector API serves to notify the maintenance function of when the medical device is idle and the estimated timeframe of idleness. The idle detector API also provides a “stop” function, i.e., if the idle detector detects that the medical device transitions from the idle state to non-idle (i.e., performing patient medical care) it can send a “stop” signal to any running maintenance functions to cause them to stop.

The following also discloses a system that includes the idle detector along with at least one maintenance function operatively connected with the idle detector by the API.

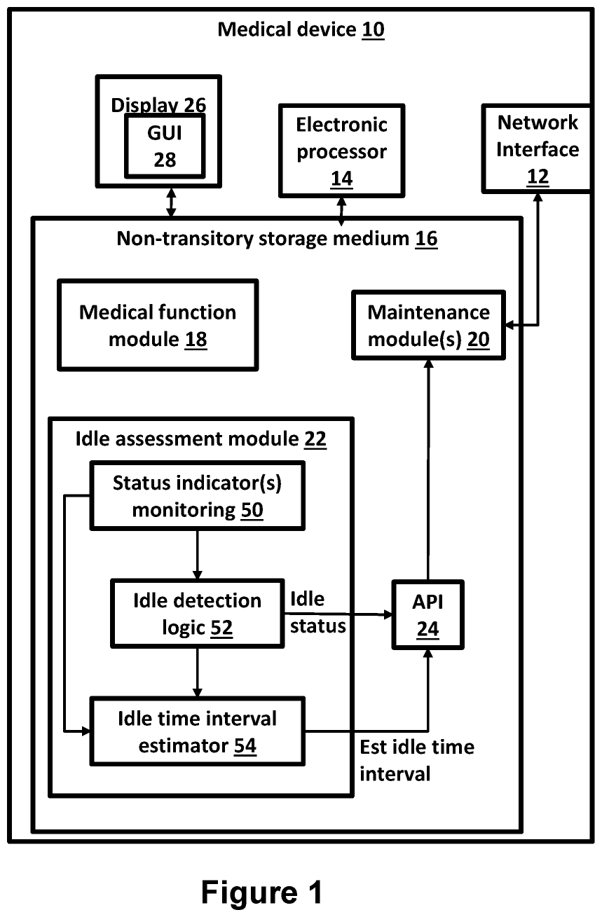

With reference to FIG. 1, an illustrative medical device 10 being monitored is diagrammatically shown. By way of some non-limiting illustrative examples, the medical device 10 being monitored may be a medical imaging device such as a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth; or may be another type of medical device such as a mechanical ventilator, a multifunction patient monitor, a radiation therapy device, or so forth.

As shown in FIG. 1, the medical device 10 includes a network interface 12 operative to connect the medical device 10 with an electronic data network (e.g., the Internet, a local area network, and so forth-not shown). For example, the network interface 12 could comprise an Ethernet card, a Wi-Fi chip or card, or so forth. The medical device 10 also includes an electronic processor 14, and a non-transitory storage medium 16 storing instructions readable and executable by the electronic processor 14 to implement one or more processing modules. Note that the medical device 10 may comprise multiple components: for example, a magnetic resonance (MR) imaging device may include the MR scanner (including the main magnet, gradient coils, radio frequency coils, and so forth) located in the magnet room and an MR scanner controller comprising the electronic processor 14 which is located in a separate, adjacent control room. In this example, the medical device 10 is considered to include both the MR scanner and the MR scanner controller.

As shown in FIG. 1, the processing module(s) can include a medical function module 18 operative to control the medical device 10 to perform patient medical care. For example, when the medical device 10 comprises a medical imaging device, the medical function module 18 is operative to control the medical imaging device 10 to acquire medical images. In another example, when the medical device 10 comprises a patient monitor, the medical function module 18 is operative to control the patient monitor 10 to acquire one or more physiological signals. In another example, when the medical device 10 comprises a mechanical ventilator, the medical function module 18 is operative to control the mechanical ventilator 10 to administer mechanical ventilation. These are merely illustrative examples and should not be construed as limiting. The medical function module 18 may also control the medical device 10 to perform ancillary functions such as loading/unloading the patient.

At least one maintenance module 20 is operative to perform at least one maintenance function for, or on, the medical device 10 utilizing the network interface 12. For example, the maintenance module(s) 20 is configured to, for example, monitor network traffic via the network interface 12 to detect network traffic anomalies; transfer, via the network interface 12, stored log data generated by the medical device 10; perform backup of data stored at the medical device 10 via the network interface 12; transfer medical data collected by the medical device 10 via the network interface 12; and so forth. These are merely illustrative examples and should not be construed as limiting.

An idle assessment module 22 is operative to determine whether the medical device 10 is in an idle state or is performing patient medical care. As used herein, the term “idle state” (and variants thereof) refers to a state of the medical device 10 when the medical device 10 is not performing patient medical care and is “not idle” when the medical device 10 is performing patient medical care. In this context, “performing patient medical care” or similar phraseology includes medical care such as acquiring patient data or delivering radiation therapy, mechanical ventilation, or other therapy to the patient. More broadly, “performing patient medical care” as used herein includes other patient/device interactions that credibly implicate patient safety, therapy efficacy, or integrity of collected patient data, such as loading a patient into a medical imaging device for imaging, unloading the patient after imaging acquisition is complete, setting up a mechanical ventilator to ventilate a patient, ventilator operations performed in conjunction with weaning the patient off the mechanical ventilator, and/or the like. Furthermore, the term “patient” as used herein encompasses any person who is receiving medical care via the medical device, whether that person is an admitted hospital patient or an outpatient or a person undergoing a screening procedure or so forth. In some examples, the idle assessment module 22 is operative to determine whether the medical device 10 is idle or is performing patient medical care based at least on automatic log entries and/or sensor readings generated by the medical device 10.

The determination of the idle state of the medical device 10 can be dependent on the type of medical device 10. For example, for medical devices that are used when actively operated (e.g., an MRI device), an activation of particular functions, menu options or device controls are indicators of non-idleness. For medical devices 10 that operate unattended (e.g., patient monitors, vital sign sensors connected to a patient) activated patient monitoring are indicators of non-idleness.

The idle assessment module 22 observes the state of the clinical function of the medical device 10, and optionally the rest of the medical device 10 (e.g., an operating system), as indicators. For example, the idle assessment module 22 is configured to read log files to determine that a medical device 10 comprising an MRI device is in part of the clinical application where no scans can be made or immediately started. In case of a patient monitor 10, the absence of connected sensors may be an indicator of clinical idleness. In another example, where the medical device 10 leverages an operating system login screen, the idle assessment module 22 can query the operating system or its logs to determine if a user is logged in for an interactive session or the medical device 10 is awaiting a user to login.

The idle assessment module 22 can include idle detection logic to determine the idleness state by taking the indicators as input. Multiple indicators can be used. By combining multiple conditions, a robust and fail-safe operation can be provided. The idle detection logic may use a rule-based approach, but also artificial intelligence (AI), analytics, machine learning, and so forth for complicated situations.

An API 24 interfaces the idle assessment module 22 and the maintenance module(s) 20 in order to control the maintenance module(s) 20 to perform the at least one maintenance function when the medical device 10 is idle and not when the medical device is performing patient medical care. For example, the medical device 10 also includes a display device 26 presenting a graphical user interface (GUI) 28 via which the medical device 10 can be controlled (e.g., via inputs provided to the display device 26). The idle assessment module 22 is operative to determine whether the medical device 10 is idle or is performing patient medical care based at least on a current user dialog of the GUI 28.

In some embodiments, the idle assessment module 22 is further operative to estimate a time interval over which the medical device 10 is expected to be idle. The API 24 interfaces the idle assessment module 22 and the maintenance module(s) 20 to control the maintenance module(s) 20 to perform the at least one maintenance function during the estimated time interval over which the medical device 10 is expected to be idle. In addition, the API 24 interfaces the idle assessment module 22 and the maintenance module(s) 20 to control the maintenance module(s) 20 to stop any currently executing maintenance function in response to the idle assessment module 22 determining a switch of the medical device 10 from idle to performing patient medical care. In this embodiment, the idle assessment module 22 comprises a machine-learning model (e.g., an artificial neural network (ANN), a decision tree, and so forth) to estimate a time interval over which the medical device 10 is expected to be in the idle state. The machine-learning model can be trained with historical data related to the idle state and the estimated time interval.

The non-transitory storage medium 16 stores instructions executable by the electronic processor 14 to perform a method of governing automated maintenance of the medical device 10. At an operation 50, the idle assessment module 22 is configured to determine one or more status indicator(s) of the medical device 10. For the example, the status indicator(s) 50 includes one or more of a user dialog of the GUI 28, automatic log entries being generated by the medical device 10, sensor readings of the medical device 10, and so forth.

At an operation 52, the idle assessment module 22 is configured to implement idle detection logic 52 to determine an idle state or status of the medical device 10. The idle status is input to the API 24.

At an operation 54, the idle assessment module 22 is configured to estimate a time interval over which the medical device 10 is expected to be in the idle state. The estimated time interval is input to the API 24. The estimation of the idle time interval can be based on various information. In one nonlimiting illustrative example, if the GUI 28 is at the login screen, then it may be known that it will take at least 3 minutes for a user to log in and initiate performance of patient medical care-hence, the idle time interval can be safely estimated as 5 minutes in this example. In another nonlimiting illustrative example, data from the medical device 10 and/or from other medical devices of the same make/model may be collected and serve as training data for training an artificial neural network (ANN) to estimate the idle time interval given as input the state of the medical device 10 as defined by the values of the status indicators determined by the monitoring 50. These are merely illustrative examples.

In general, the operation 54 should be conservative in estimating the idle time interval, i.e., the actual idle time interval should usually be longer than the estimated idle time interval. However, as will be discussed, the idle assessment module 22 can detect if the medical device 10 transitions from the idle state to the non-idle state while a maintenance function is being performed and can trigger the maintenance function to abort in such a case. Hence, it is acceptable if occasionally the actual idle time interval is shorter than the estimated idle time interval.

When the idle state of the medical device 10 is idle (i.e., not performing patient medical care), the API 24 is configured to interface with the maintenance module(s) 20 to perform a maintenance function process for, or on, the medical device 10 within the estimated time interval. In some examples, the idle assessment module 22 is configured to determine that the idle state of the medical device 10 has changed from idle to not idle (i.e., the medical device 10 has begun to perform patient medical care). When this happens, the API 24 is configured to interface with the maintenance module(s) 20 to stop the maintenance function process.

In some embodiments, the API 24 is configured to interface with the maintenance module(s) 20 and the idle assessment module 22 to make a request to perform the maintenance function process. The request can specify if it is a request for immediate or scheduled execution, indication of execution duration (optional) and callback endpoint. The idle assessment module 22 responds with idle or non-idle for immediate requests, or registered or non-registered for scheduled requests.

The idle assessment module 22 may use the duration indication in its idle detection logic to evaluate the request, e.g., predict idleness chance for indicated duration. This offers the additional benefit of avoiding overhead of starting and stopping maintenance functions, i.e., try to finish the maintenance function within the expected duration of idleness.

The idle assessment module 22 registers a callback endpoint for all non-rejected requests. For immediate requests with idle response, the idle assessment module 22 invokes a callback endpoint to indicate change of the idle state to non-idleness. For scheduled requests, the idle assessment module 22 invokes the callback endpoint to signal the start and end of idleness.

When the idle assessment module 22 receives the request response or callback it starts, pauses, stops, etc., the maintenance function. In an extension, the idle assessment module 22 implements a failsafe where it stops the maintenance function process when not paused or stopped within a pre-defined time.

Maintenance functions include network security monitoring, network security scanning, log collection and upload, incremental backups, software integrity scans, medical device configuration checks, and/or so forth. Such maintenance functions may involve significant resources, e.g., network security monitoring may perform real-time analysis of network traffic to detect anomalies. Another example is large backups, e.g., full system backup or backup of all image data.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

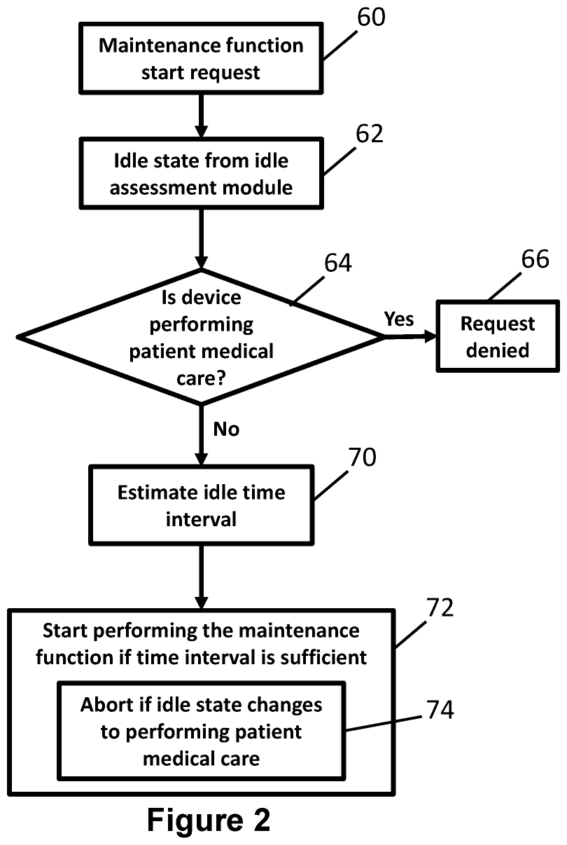

With continuing reference to FIG. 1 and further reference to FIG. 2, an illustrative method of regulating performance of a maintenance function using the apparatus of FIG. 1 is described. In an operation 60, a maintenance function start request is received at the API 24 from one of the maintenance modules 20. In an operation 62, the idle assessment module 22 determines the idle state. At an operation 64, it is determined whether the idle state indicates the medical device 10 is performing patient medical care. If the device is performing patient medical care, then the request is denied at operation 66, for example by the idle assessment module 22 sending a denial back to the requesting maintenance module 20 via the API 24.

On the other hand, if at the decision block 64 it is determined that the medical device 10 is idle (that is, is not currently performing patient medical care), then flow passes to an operation 70 where the idle time interval is estimated by the idle assessment module 22. This information (i.e., that the medical device is currently idle and is expected to remain idle for the estimated idle time interval) is sent to the requesting maintenance module 20 via the API 24, which then in an operation 72 starts performing the maintenance function if the estimated time interval (from operation 70) is sufficient to do so.

During the operation 72, the idle assessment module 22 continues to monitor the idle state. If, while the maintenance function is being performed, the idle assessment module 22 determines that the medical device 22 has transitioned from idle to non-idle (i.e., has begun performing patient medical care) then in an operation 74 the idle assessment module 22 sends an abort signal to the requesting maintenance module 20 via the API 24. The requesting maintenance module 20 then aborts the in-progress maintenance function. In some variant embodiments, this aborting may be a “soft” abort, for example, the maintenance module 20 may perform a shutdown of the in-progress maintenance function that saves the state of the in-progress maintenance function so it can be restarted and completed at a later time. Such a soft shutdown is preferably fast and/or utilizes few microprocessor resources so that it does not interfere with the performance of the patient medical care.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions readable and executable by at least one electronic processor to perform a method of governing automated maintenance of a medical device, the method comprising:
determining at least one status indicator of a medical device;
determining, from the at least one status indicator, an idle state of the medical device; and
when the idle state of the medical device is idle, performing a maintenance function on the medical device.

2. The non-transitory computer readable medium of claim 1, wherein the at least one status indicator includes one or more of a user dialog of a graphical user interface (GUI) currently being displayed on the medical device, automatic log entries being generated by the medical device, and sensor readings of the medical device.

3. The non-transitory computer readable medium of claim 1, wherein the method further includes:
estimating a time interval over which the medical device is expected to be in the idle state.

4. The non-transitory computer readable medium of claim 3, wherein the estimating is performed by a machine-learning model.

5. The non-transitory computer readable medium of claim 3, wherein the method further includes:
training the machine-learning model with historical data related to the idle state and the estimated time interval.

6. The non-transitory computer readable medium of claim 3, wherein the method further includes:
performing the maintenance function process within the estimated time interval.

7. The non-transitory computer readable medium of claim 6, wherein the method further includes:
determining that the idle state of the medical device has changed from idle to not idle; and
stopping the maintenance function process in response to the determination that the idle state of the medical device has changed from idle to not idle.

8. The non-transitory computer readable medium of claim 1, wherein the idle state is idle when the medical device is not performing patient medical care and is not idle when the medical device is performing patient medical care.

9. A medical device, comprising:
a network interface operative to connect the medical device with an electronic data network;
an electronic processor; and
a non-transitory storage medium storing instructions readable and executable by the electronic processor to implement processing modules including:
a medical function module operative to control the medical device to perform patient medical care;
at least one maintenance module operative to perform at least one maintenance function on the medical device utilizing the network interface;
an idle assessment module operative to determine whether the medical device is idle or is performing patient medical care; and
an application programming interface (API) interfacing the idle assessment module and the at least one maintenance module to control the at least one maintenance module to perform the at least one maintenance function when the medical device is idle and not when the medical device is performing patient medical care.

10. The medical device of claim 9, wherein the medical device is one of:
a medical imaging device, wherein the medical function module is operative to control the medical imaging device to acquire medical images;
a patient monitor, wherein the medical function module is operative to control the patient monitor to acquire one or more physiological signals;
a mechanical ventilator, wherein the medical function module is operative to control the mechanical ventilator to administer mechanical ventilation; or
a radiation therapy device, wherein the medical function module is operative to control the radiation therapy device to deliver therapeutic radiation.

11. The medical device of claim 9, wherein the at least one maintenance module operative to at least one of:
monitor network traffic via the network interface to detect network traffic anomalies;
transfer, via the network interface, stored log data generated by the medical device;
perform backup of data stored at the medical device via the network interface; and/or
transfer medical data collected by the medical device via the network interface.

12. The medical device of claim 9, further comprising:
a display presenting a graphical user interface (GUI) via which the medical device can be controlled;
wherein the idle assessment module is operative to determine whether the medical device is idle or is performing patient medical care based at least on a current user dialog of the GUI.

13. The medical device of claim 9, wherein the idle assessment module is operative to determine whether the medical device is idle or is performing patient medical care based at least on automatic log entries and/or sensor readings generated by the medical device.

14. The medical device of claim 9, wherein:
the idle assessment module is further operative to estimate a time interval over which the medical device is expected to be idle; and
the API interfaces the idle assessment module and the at least one maintenance module to control the at least one maintenance module to perform the at least one maintenance function during the estimated time interval over which the medical device is expected to be idle.

15. The medical device of claim 9, wherein the API interfaces the idle assessment module and the at least one maintenance module to control the at least one maintenance module to stop any currently executing maintenance function in response to the idle assessment module determining a switch of the medical device from idle to performing patient medical care.

16. A method of governing automated maintenance of a medical device, the method comprising:
determining at least one status indicator of a medical device;
determining, from the at least one status indicator, an idle state of the medical device, wherein the idle state is idle when the medical device is not performing patient medical care and is not idle when the medical device is performing patient medical care; and
preventing a maintenance function on the medical device from being performed when the idle state of the medical device is not idle.

17. The method of claim 16, wherein the at least one status indicator includes one or more of a user dialog of a graphical user interface (GUI) currently being displayed on the medical device, automatic log entries being generated by the medical device, and sensor readings of the medical device.

18. The method of claim 16, further including:
estimating a time interval over which the medical device is expected to be in the not idle state.

19. The method of claim 18, wherein the estimating is performed by machine-learning model.

20. The method of claim 16, further including:
determining that the idle state of the medical device has changed from not idle to idle; and
performing the maintenance function process in response to the determination that the idle state of the medical device has changed from not idle to idle.
